(19) United States
(12) Patent Application Publication
(10) Pub. No.: US 2003/0073091 A1
(43) Pub. Date: Apr. 17, 2003

(54) Use of generic oligonucleotide microchips to detect protein-nucleic acid interactions

(75) Inventors: Alexander S. Krylov, Moscow (RU); Andrei Mirzabekov, Moscow (RU); Dmitry V. Prokopenko, Moscow (RU); Olga A. Zasedateleva, Moscow (RU)

Correspondence Address: FOLEY & LARDNER, 150 EAST GILMAN STREET, P.O. BOX 1497, MADISON, WI, 53701-1497, US

(73) Assignee: The University of Chicago

(21) Appl. No.: 10/035,042

(22) Filed: Dec. 27, 2001

Related U.S. Application Data
(60) Provisional application No. 60/258,824, filed on Dec. 28, 2000.

(51) Int. Cl. C12Q 1/68

(52) U.S. Cl. 435/6

(57) ABSTRACT

Nucleic acids or proteins immobilized in a gel pad are interacting with a protein and the nucleic acid-protein and protein-protein interactions are characterized and measured. Large-scale, parallel measurements of these interactions can be examined to provide a powerful tool in elucidating interactions between proteins and nucleic acids.

### CLAIM OF PRIORITY

This application claims priority from U.S. Provisional Patent Application No. 60/258,824, filed Dec. 28, 2000, the entire contents of which are hereby incorporated by reference.

### STATEMENT REGARDING FEDERALLY SPONSORED RESEARCH

This invention was made with Government support under Contract No. W-31-109-ENG-38 awarded by the Department of Energy. The Government has certain rights in this invention.

### FIELD OF INVENTION

The present invention relates to methods for measuring protein-nucleic acid and protein-protein interactions. More particularly, the present invention provides methods and kits for measuring the strength of these interactions.

### BACKGROUND OF THE INVENTION

The interaction between proteins and nucleic acids plays a fundamental role in virtually every cellular event, particularly in gene regulation and nucleic acid replication. However, the interactions between proteins and nucleic acids are not well understood or easily predicted.

Different methods have been used to study these interactions. For example, binding small ligands with DNA has been studied by several well-characterized techniques, such as protection of nucleic acids in a complex against chemical modifications, nuclease footprinting assays, separation of the complexes by electrophoresis, dialysis and optical methods in the case of small ligands. Immobilization of oligonucleotides on filters or glass surfaces also provides a means to assay protein-DNA interactions. All of these methods are usually applied to discriminate stringent specific binding from nonspecific binding, and these findings usually require painstaking research in order to determine the nucleic acid sequence for which the protein has the highest specificity and/or affinity. Nucleic acid binding proteins have been discovered that interact only with single (ss)DNA or double stranded (ds)DNA, or RNA and these proteins often have different degrees of DNA or RNA sequence specificity. For example, the specific binding of the Cro repressor to its active site is 10⁸ times stronger than the nonspecific binding, the binding constant of Hoechst 33258 to AT-rich sequences is 10³ times higher than that to GC-rich sequences. However, it is difficult, it not impossible, to find ‘soft’ specificities when the binding constants of the protein or small ligands to all sequences is of the same order of magnitude.

Thus, there continues to be a need to readily characterize the interactions between nucleic acids and proteins.

### SUMMARY OF THE INVENTION

Discussed herein are methods for characterizing and measuring the interactions between proteins and other proteins or nucleic acids. According to these methods, a protein or nucleic acid is immobilized on a solid support, for example a gel pad, and the nucleic acid or proteins are contacted so that they interact with one another. The strength of the interaction, if any, is then measured providing a characterization of the interaction. Multiple iterations of this method can also be performed, simultaneously or subsequent to other iterations. Fluorescence and melting temperature, or changes therein, are two useful ways to measure the strength of the protein-protein or nucleic acid-protein interaction. In some aspects, the identity and sequence of the nucleic acid, proteins, or both are known, whereas in others the identity of one or more of these is not known and can later be determined as desired. All nucleic acids and proteins can be used in the present methods, including functional nucleic acids coding for a promoter or an entire gene(s), and functional proteins, for example those that modulate the expression of a gene or activity of a gene product. Kits for carrying out these methods are also disclosed.

Objects and advantages of the present invention will become more readily apparent from the following detailed description.

### BRIEF DESCRIPTION OF THE DRAWINGS

FIG. 1 shows non-equilibrium melting curves for a microchip duplex measured in the absence and presence of HU protein. A duplex was formed by hybridization of the oligonucleotides gel-MAGTCTGM-3′ from the gel-pad with the oligonucleotides 5′-MTCAGACM-5′-TR from the hybridization mixture. Non-equilibrium melting temperature Tm was defined as described in Materials and Methods. The HU protein affinity to the duplex was measured as ΔTm=Tm(HU)−Tm(A);

FIG. 2 is a histogram showing the number of duplexes N demonstrating specified ΔTm. There are nearly 800 duplexes with a positive ΔTm and 200 with a negative one;

FIG. 3 (A) shows average shifts of Tm for all the duplexes with two bases motifs. The first 7 motifs are presented. 3 (B) shows average shifts of Tm for all the duplexes with three bases motifs. The first 7 motifs are presented;

FIG. 4 is a plot of fluorescent signals from the duplexes formed with the protein against the signals from free duplexes. G/C-rich duplexes are dark gray; A/T-rich are black; the “intermediate” ones are light gray;

FIG. 5 illustrates the dependence of signal ratio (with protein/without protein) on the temperature shifts of duplexes with the protein. The diagram indicates that A/T-rich sequences (black) give less intense signals and negative Tm values;

FIG. 6 depicts non-equilibrium melting curves for the complexes of FITC-labeled HU protein with several immobilized octamers. The general structure of the immobilized octamers is gel-MNNNNNNM-3′, where NNNNNN is the hexamer core and M are the flanking bases. The 5 curves with different hexamer cores are presented; and

FIG. 7 (A) shows average melting temperatures for the duplexes with different numbers of G/C bases in the hexamer core. 7 (B) shows average intensity of fluorescence signal for the duplexes with different numbers of G/C.

### DETAILED DESCRIPTION OF PREFERRED EMBODIMENTS

One embodiment of the present invention provides a method for measuring the interaction between nucleic acids and proteins. According to this method a nucleic acid is immobilized on a solid support, such as gel pad, interacted with a protein and the strength of the interaction between the protein and nucleic acid is measured. Alternatively, the protein can be immobilized on the solid support instead of the nucleic acid. Suitable nucleic acids useful in the present methods include DNA, both single-stranded and double-stranded, RNA, both single-stranded and double-stranded and including mRNA (messenger), tRNA (transfer), rRNA (ribosomal), snRNA (small nuclear), snoRNA (small nucleolar), scRNA, hnRNA (heteronuclear), and nucleic acid mimics, such as peptide nucleic acid (PNA) which replaces the nucleic acid sugar-phosphate backbone with a pseudopeptide backbone. The nucleic acid can either be functional, such as a gene, promoter, terminator, or the like, or nonfunctional, as desired. Nucleic acids used in subsequent iterations of the present invention can be related to the first nucleic acid, such as where the other nucleic acids have mutations of the first nucleic acid at one or more positions. The nucleic acid can be of any desired length and can be extremely short or long depending upon the desired application. Nucleic acid sequences can be short enough such that they lack secondary structure. In fact, the present invention can be used with nucleic acids whose sequences are undetermined, but are subsequently determined by interaction with the protein or by conventional techniques, such as using nucleic acid probes or sequencing analysis. The nucleic acid can be isolated from a particular source, synthesized or amplified as desired.

When double-stranded nucleic acids are used in the present methods, the nucleic acids can be hybridized under varying stringency conditions. The terms, high stringency, medium stringency, low stringency and the like encompass meanings well known to those in the art. Generally, “highly stringent conditions” describes conditions which require a high degree of matching to properly hybridize nucleic acids, which typically occurs under conditions of low ionic strength and high temperature. The expression “hybridize under low stringency” commonly refers to hybridization conditions having high ionic strength and lower temperature.

Variables affecting stringency include, for example, temperature, salt concentration, probe/sample homology, nucleic acid length and wash conditions. Stringency is increased with a rise in hybridization temperature, all else being equal. Increased stringency provides reduced non-specific hybridization. i.e., less background noise. “High stringency conditions” and “moderate stringency conditions” for nucleic acid hybridizations are explained in Current Protocols in Molecular Biology, Ausubel et al., 1998, Green Publishing Associates and Wiley Interscience, NY, the teachings of which are hereby incorporated by reference. Of course, the artisan will appreciate that the stringency of the hybridization conditions can be varied as desired, in order to include or exclude varying degrees of complementation between nucleic acid strands, in order to achieve the required scope of detection. Likewise the protein and nucleic acid can be interacted under varying conditions which either enhance or interfere with protein-nucleic acid interactions.

Similarly, the protein capable of being used in the present invention is not limited. For example, proteins can be used which bind nonspecifically to a nucleic acid or to a specific nucleic acid sequence, such as proteins which regulate gene expression and/or activity. The protein can either be a functional protein or a protein fragment. Proteins can also be simple proteins, which are composed of only amino acids, and conjugated proteins, which are composed of amino acids and additional organic and inorganic groupings, certain of which are called prosthetic groups. Conjugated proteins include glycoproteins, which contain carbohydrates; lipoproteins, which contain lipids; and nucleoproteins, which contain nucleic acids. As above, the identity of the protein need not be known when interacted with the nucleic acid and can be determined at a later point through known techniques, In fact, the present invention can be used to identify novel proteins and characterize their interactions with nucleic acid. Different proteins can also be used in different iterations of the present method using the same nucleic acid. Related proteins can also be used in these iterations to determine the effect mutations in the protein have on the measured interactions. Likewise, proteins having a known mutation can be tested in parallel with the wild-type protein to determine the possible effects the protein mutation has on nucleic acid-protein interactions.

One typical protein known to bind nonspecifically to double-stranded DNA (ds DNA) is the bacterial HU protein. It is an abundant (30,000 dimers per cell), small (18 kDa), basic, and heat-stable protein associated with the bacterial nucleoid in Escherichia coli. The HU protein is composed of two very homologous polypeptides, and the heterodimeric form, is predominant during stationary phase. This protein has the capacity to introduce in vitro negative supercoils in relaxed circular DNA in the presence of topoisomerase 1 and to condense DNA. HU binds to both double-stranded and single-stranded DNA (ss DNA), and to some other structural forms of DNA. The binding of HU protein to ds DNA is known to be sequence-nonspecific, and the specificity of binding to ss DNA has not been described yet.

Generally, the present method involves immobilizing either the nucleic acid or protein on a solid support and interacting the protein and nucleic acid by contacting them with each other. This process is preferably repeated one or more times using nucleic acids with different sequences or different proteins. Accordingly, the presence or absence of protein-nucleic acid interaction can be easily measured, as well as the strength of any interaction. Any suitable method for immobilizing the nucleic acid on the solid support can be used in the present invention. Immobilization techniques can occur through chemical coupling, such as by reductive coupling, and include those disclosed in Timofeev, E. et al., (1996) Nucleic Acids Res., 24, 3142-3148 and U.S. Pat. No. 5,981,734. Additional methods for linking molecules (e.g., polypeptides and polynucleotides) to solid phases are well known and include methods used for immobilizing reagents on solid phases for solid phase binding assays or for affinity chromatography (see, e.g., chapter 9 of Immunoassay, E. P. Diamandis and T. K. Christopoulos eds., Academic Press: New York, 1996, and Hermanson, Greg T., Immobilized Affinity Ligand Techniques, Academic Press: San Diego, 1992). These methods include the non-specific adsorption of molecules on the reagents on the solid phase as well as the formation of a covalent bond between the reagent and the solid phase. Alternatively, a substrate can be linked to a solid phase through a specific interaction with a binding group present on the solid phase (e.g., an antibody against a peptide substrate or a nucleic acid complementary to a sequence present on a nucleic acid substrate). In an advantageous embodiment, a substrate or product labeled with a binding reagent A (also referred to as a capture moiety) is contacted with a second binding reagent B present on the surface of a solid phase, so as to link the substrate to the solid phase through an A:B linkage.

Preferred methods involve immobilizing the nucleic acid or protein on a substrate which closely simulate solution conditions, such as substrates including a buffer solution, such as a gel, for example agarose, dimethylacrylimide or polyacrylamide. More preferably, the methods utilize a substrate for which there is a direct correlation exists between the thermodynamic parameters of nucleic acids and proteins in the substrate as compared to solution, such as a microchip gel pad. Fotin, A. V. et al., (1998) Nucleic Acids Res., 26, 1515-1521. Gel-pad microchips containing immobilized oligonucleotides provide some essential advantages over the microchips based on glass or filters as gel-pad microchips have a higher capacity and provide more homogeneous environment for hybridization, and as such the terms “solid support” or “substrate” used in the present invention specifically exclude glass and filters.

When used, the gel-pad chip preferably has at least an array of 100 (10×10) gel pads and more preferably an array of at least 1000 gel pads. Accordingly, a large number of samples can be simultaneously tested. Preferably, hundreds, if not thousands, of such reactions are carried out simultaneously. Likewise, only a minute amount of protein or nucleic acid is required for each gel pad, such as is present in one to ten nanoliters of a 0.1 to 100 mM solution. Surprisingly and unexpectedly, meaningful data can be obtained utilizing these infinitesimal amounts of protein and/or nucleic acid.

Preferably, either the nucleic acid, protein or both are labeled. Suitable labels include ligands which bind to labeled antibodies, fluorophores, chemiluminescent agents, enzymes, and antibodies which can serve as specific binding pair members for a labeled ligand. Fluorescence quenching labeling schemes can also be used in the present methods, wherein one of the protein or nucleic acid is labeled with a fluorescent moiety and the other is labeled with a quenching moiety such that interaction of the two results in fluorescent quenching. One or more labels can also be incorporated onto the nucleic acid and/or protein. This can be useful when a nucleic acid of significant length us used in order to determine where the protein interacts with the nucleic acid. Multiple labels on the protein can also provide and indication about which part of the protein interacts with the nucleic acid.

The label may also allow for the indirect detection of the hybridization complex. For example, where the label is a hapten or antigen, the sample can be detected by using antibodies. In these systems, a signal is generated by attaching fluorescent or enzyme molecules to the antibodies or, in some cases, by attachment to a radioactive label. (Tijssen, “Practice and Theory of Enzyme Immunoassays,” Laboratory Techniques in Biochemistry and Molecular Biology” (Burdon, van Knippenberg (eds.), Elsevier, pp. 9-20 (1985)).

The detectable label used in nucleic acids of the present invention may be incorporated by any of a number of means well known to those of skill in the art. However, in a preferred embodiment, the label is simultaneously incorporated during the synthesis or amplification step in the preparation of the sample nucleic acids. Thus, for example, polymerase chain reaction (PCR) with labeled primers or labeled nucleotides will provide a labeled amplification product. In another preferred embodiment, transcription amplification using a labeled nucleotide (e.g. fluorescein-labeled UTP and/or CTP) incorporates a label into the transcribed nucleic acids.

Alternatively, a label may be added directly to an original nucleic acid sample (e.g., mRNA, polyA mRNA, cDNA, etc.) or to the amplification product after the amplification is completed. Means of attaching labels to nucleic acids are well known to those of skill in the art and include, for example nick translation or end-labeling (e.g. with a labeled RNA) by phosphorylation of the nucleic acid and subsequent attachment (ligation) of a nucleic acid linker joining the sample nucleic acid to a label (e.g., a fluorophore).

Useful labels in the present invention include biotin for staining with labeled streptavidin conjugate, fluorescent dyes (e.g., fluorescein, texas red, rhodamine, green fluorescent protein, and the like), radiolabels (e.g., ³H, ¹²⁵I, ³⁵S, ¹⁴C, and ³²P), and enzymes (e.g., horse radish peroxidase, alkaline phosphatase and others commonly used in an ELISA). Patents teaching the use of such labels include U.S. Pat. Nos. 3,817,837; 3,850,752; 3,939,350; 3,996,345; 4,277,437; 4,275,149; and 4,366,241.

Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label.

The interaction between the nucleic acid and protein can be characterized by any means known in the art. Preferably, the interaction is characterized by measuring an event which causes or quenches fluorescence. Alternatively, the strength of the interaction can be determined by measuring the melting temperature of the nucleic acid or the temperature which causes dissociation of the protein from the nucleic acid.

Thus, the present methods provide for extremely high throughput. For example, thousands, if not tens of thousands, of samples can be simultaneously tested in a matter of minutes. In one embodiment, fluorescence microscopy is used for quantitative, real-time measurement of the interaction of nucleic acid protein interactions which are fluorescently labeled.

Surprisingly and unexpectedly, the present invention has been found to elicit preferential binding motifs for proteins which were thought to bind nucleic acids in a non-preferential manner.

The methods of the present invention are also readily suitable for studying protein-protein interaction through modifications which will be readily apparent to one of skill in the art. In this embodiment of the present invention one of the proteins is immobilized on a substrate and reacted with the second protein. The present invention is also capable of being easily modified to characterize the interactions between nucleic acids and non-protein substances, for example salts, small organic molecules and the like. In a similar vein the present invention can be used to study interactions between two or more proteins and a nucleic acid.

In a further embodiment of the invention the interaction between a protein and a nucleic acid or a protein and a protein can be characterized in the presence of one or more test agents to determine what effect, if any, the test agent has on the interaction. After a test agent is identified as having a desired property, the test agent can be identified and either isolated or chemically synthesized to produce a therapeutic drug. Thus, the present methods can be used to make drug products useful for therapeutic treatment both in vitro and in vivo. The test agent can be applied by any means well known in the art, such as by adding the test agent to the buffer solution making up the gel-chip or adding the test agent after interaction of the other components has occurred. Generally, this embodiment will involve interacting the proteins or nucleic acids as described above in the presence of the test agent and comparing the protein-nucleic acid or protein-protein interaction against a control lacking the test agent. This embodiment can be used to find lead compounds which can be modified in an effort to find more effective drugs.

The present invention also provides kits for carrying out the methods described herein. In one embodiment, the kit is made up of instructions for carrying out any of the methods described herein. The instructions can be provided in any intelligible form through a tangible medium, such as printed on paper, computer readable media, or the like. The present kits can also include one or more reagents, buffers, hybridization media, gel chips, chromatic or fluorescent dyes and/or disposable lab equipment, such as multi-well plates in order to readily facilitate implementation of the present methods.

In another embodiment, nucleic acid sequencing and identification can be performed by interacting a nucleic acid with a protein or proteins known to have a high specificity for a specific nucleic acid sequence. Strong interaction of the protein with the nucleic acid will indicate that the nucleic acid has the sequence for which the protein is specific. The sequence of the nucleic acid can then be confirmed through other means, such as sequencing. Likewise, using a nucleic acid with a known sequence can be used to identify proteins which bind preferentially with that sequence. In this embodiment, the nucleic acid sequence is known and proteins which strongly bind with the sequence can be isolated and identified. In this manner targets for drug therapy can be identified to enhance or disrupt these interactions. These embodiments can also be used to purify the bound nucleic acid or protein. According to this method, once bound, impurities or contaminants can be washed off the solid support, the interaction between the protein and nucleic acid can be disrupted and the nucleic acid or protein washed off to provide a purified nucleic acid or protein.

As illustrated above, the methods of the present invention have a wide variety of uses that will be readily apparent to a person having ordinary skill in the art including at least:

Diagnostic utilities for diseases caused by nucleic acid-protein interactions and protein-protein interactions;

Drug discovery, testing, resistance analysis and lead compound discovery;

Regulation of gene expression;

Determining the sequence of nucleic acids including DNA typing;

Isolation of nucleic acid sequences and/or proteins;

Nucleic acid and protein binding analysis;

Determining the identity of proteins;

Measuring sequence specificity of nucleic acids and proteins, specifically measuring the effect of mutations thereon; and

Identifying new proteins which interact, and modulate, genes and gene products.

This invention is further illustrated by the following non-limiting examples.

### EXAMPLES

### Example 1

In the present example, a generic microchip was used for a large-scale parallel analysis of the HU binding to different 8mer duplexes containing variable 6mer cores. This type of microarray provided a homogeneous environment for protein-DNA binding close to conditions in solution. It also enabled the study of more than 1000 melting curves of the DNA duplexes in the absence or presence of HU protein, and the statistical analysis was applied to find those motives, which are preferable for binding. These statistics uncovered the “hidden” specificity of HU protein-DNA binding.

Large-scale parallel measurements of the melting curves of 1024 octamer duplexes on a generic microchip in the absence or presence of HU protein is described. The generic microchip contained all possible 4,096 hexadeoxynucleotide sequences flanked at the 3′ and 5′ ends with a nucleotide represented a mixture of four bases. The resulting octamers were chemically immobilized inside polyacrylamide gel pads. After that, 1024 selected octamers were converted to the double-stranded (ds) form by hybridization with a mixture of fluorescently labeled complementary octamers. The statistical investigation of 1024 melting curves of the octamers in the absence or presence of HU provided information on the stability of protein-DNA complexes. It is shown that, in regards to the melting temperature shift, the octamer duplexes can be divided into two groups: the major one (85%), which is characterized by the Tm increase for the complexes compared with the duplexes, and the minor one, where the Tm decrease for the complexes was observed. In the major group, the HU-ds DNA complex displayed no stringent specificity. However, for some sequence motifs, e.g., AA, AAG, or AGA, the HU binding stabilized ds DNA. A correlation has been found between Tm of HU-DNA complexes and the quenching of octamer duplex fluorescence by HU. In a second set of experiments, the binding of fluorescein-labeled HU protein with the single-stranded (ss) DNA was studied. A moderate preferential HU binding with G/C-rich sequences was observed. The results are discussed in regards to the pleiotropic role played by HU in the bacterial cells and demonstrate the possibility of using microchips as a powerful tool to study protein-DNA interactions.

The results demonstrate that the binding of HU protein to ds DNA has no stringent specificity, but surprisingly and unexpectedly some DNA motifs are bound preferentially. It was also found that HU can preferentially bind to AT-rich ss DNA sequences. These results demonstrate that gel-pad generic microchips can be used to study nucleic acid-protein interactions.

### MATERIALS AND METHODS

Chemicals

4,096 octadeoxyribonucleotides used for the manufacturing of generic microchips were purchased from CyberSyn (USA). These 8mers have the structure 5′-NH2-MNNNNNNM-3′, where M is 1:1:1:1 mixture of the four bases at the both 3′ and 5′ terminal positions; N is one of the four bases of the core representing in total 4096 possible 6mers; NH2 is an amino-linker used to immobilize the 8mers to the polyacrylamide gel pads of the microchips. The 8mer mixture 5′-MM(A/C)MM(A/C)MM-NH2-3′ was synthesized with an Applied Biosystems 394 DNA/RNA synthesizer using standard phosphoramidite chemistry and 3′-C(7) amino modifier CPG (Glen Research, USA). The 8mer mixture was fluorescently labeled with Texas Red (TR) sulphonyl chloride dye (Molecular Probes, Eugene, Oreg.) according to the manufacturer's protocol.

Generic Microchips

The generic microchips were manufactured in two steps. First, arrays of 4200 (60×70) 5% polyacrylamide gel pads (100×100×20 μm spaced by 200 μm) were prepared by photopolymerization as discussed in Timofeev, E. et al. (1996) Nucleic Acids Res., 24, 3142-3148. Then, one-nanoliter droplets of 1 mM solutions of oligonucleotides in water were applied to each gel pad on a hydrophobic glass slide (Yershov, G., et al. (1996) Proc. Nat. Acad. Sci. USA, 93, 4913-4918) and the oligonucleotides were immobilized by reductive coupling of their amino groups with aldehyde groups of the gel.

HU Protein

Native HU αβ protein was purified from E. coli strain JRY1 as described in Rouviere-Yaniv, J. and Kjeldgaard, N. O. (1979) FEBS Letters, 106, 297-300 with some improvements to remove nuclease activity, which is strongly associated with HU. The protein concentration was determined from absorbance at 230 nm, where A230=2.3 corresponds to 1 mg/ml of HU protein.

For the experiments with ss DNA, HU protein was labeled with FITC in accordance with the standard protocol discussed in Guschin, D., et al. (1997) Anal. Biochem., 250, 203-211 in a Na-carbonate buffer pH=9.3 containing 0.15M NaCl:FITC was added to the protein solution (30 μg/mg of protein). The mixture was incubated for 1.5 h at room temperature, and then FITC was removed from the labeled protein by gel filtration on Sephadex G-25.

Hybridization and Melting Measurements

Hybridization of the generic microchip with the mixture of fluorescently labeled 6mers was carried out in a 200-μl hybridization chamber at 0° C. for 24 h. The hybridization solution contained 200 μM oligonucleotides, 100 mM NaCl, 20 mM Tris (pH 7.2), 5 mM EDTA, and 0.1% Tween 20. After hybridization, the solution was replaced with the same buffer without oligonucleotides. The hybridization chamber with the microchip was then placed on the thermotable of fluorescence microscope and the melting curves were recorded for all the elements of the microchip. The temperature increase was from −2° C. to +50° C. at the rate of 2° C./h in 1° C. steps. After measuring the melting curves of the duplexes in the absence of HU protein, the fluorescently labeled oligonucleotides were washed off the microchip with water. A second round of hybridization and melting experiments was performed under the same conditions, but this time the solution was replaced with a buffer containing HU protein (0.55 mg/ml) and incubated for 12 hours at 0° C. Then the same melting procedure was performed.

All measurements of the melting curves were performed using the automated 3.5×3.5-mm field epifluorescent microscope with mercury lamp excitation and a filter for Texas Red dye (LOMO, Russia). The microscope was equipped with a CDD camera (Princeton Instruments, USA), a Peltier thermotable with a temperature controller (Melcor, USA), and a computer supplied with a data acquisition board (National Instruments, USA). The fluorescence intensity was measured at each temperature by scanning the generic microchip by fields containing 100 gel pads. To acquire an image of 100 pads took 2 sec. The scanning system consisted of a two-coordinate table, stepped motors, and a controller (Newport, USA). Special software was designed for experimental control and data processing using the C++ or the LabVIEW virtual instrument interface (National Instruments, USA).

Results

Large-scale parallel measurements of HU protein-oligonucleotide interactions on generic microchip

The generic 6mer microchip contains all possible 4,096 single-stranded hexadeoxyribonucleotides NNNNNN (N, one of four bases). These core 6mers are flanked within 8mers of the general structure gel-5′-MNNNNNNM-3′ from both 3′ and 5′ ends with 1:1:1:1 mixture of four bases, M. The resulted 8mers are immobilized within gel pads; each gel-pad contains only one 6mer.

HU protein is known to bind ds DNA but no significant sequence specificity was observed. However the specificity of HU protein-DNA complexes was reexamined by statistical analysis of large-scale data on duplex melting curves. To perform such measurements, the single-stranded oligonucleotides on the generic microchip were converted to the double-stranded ones. This was achieved by hybridization of the microchip with a mixture of fluorescently labeled 8mers of the similar structure 5′-MNNNNNNM-3′-TR. To avoid competitive oligonucleotide hybridization between the solution and the microchip, the mixture containing 1,024 different noncomplementary oligonucleotides labeled with Texas Red (TR) was synthesized according to the formula: 5′-MM(A/C)MM(A/C)MM-3′-NH2-TR.

After hybridization with fluorescently labeled 8mers and washing (see Materials and Methods), nonequilibrium melting curves for all duplexes formed on the microchip were recorded at increasing temperature. For the second stage of the experiment, the hybridization and recorded the melting curves on the same microchip were repeated, however, this time, the incubation was performed in the presence of HU protein to allow formation of the protein-oligonucleotide complexes. The melting curves were obtained in exactly the same way as in the absence of HU protein.

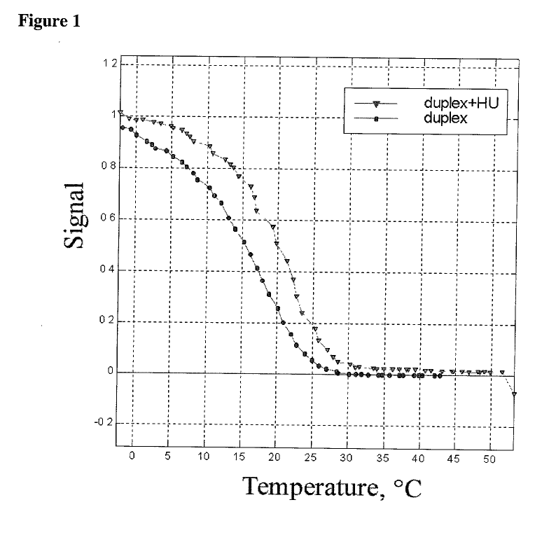

FIG. 1 demonstrates, as an example, two such melting curves obtained for the same oligonucleotide AGTCTG. A special computer program was used to calculate the difference in melting temperatures (ΔTm) between duplexes in the presence or absence of HU protein. All the 1,024 melting curves were approximated by least squares method with the following equation:

$$f ( T ) = A + \frac{B}{1 + {( \frac{T}{{T}_{0}} )}^{N}} , \qquad ( 1 )$$

where T is the temperature (° K); f(T), signal measured; T₀, the melting temperature; A+B, the initial signal; B, the fmal signal, N, cooperativity factor. When the approximation was done, 1,024 Tm values for the melting curves in the absence of HU protein and 1,024 Tm values for the melting curves in the presence of HU protein were obtained. The total overall ΔTm=Tm(protein)−Tm(free) for all the duplexes was also obtained. Fourteen oligonucleotides were excluded from the consideration owing to a weak hybridization signal. A total of 1,010 values of ΔTm were subjected to statistical analysis.

Analysis of HU Binding Motifs in Duplexes

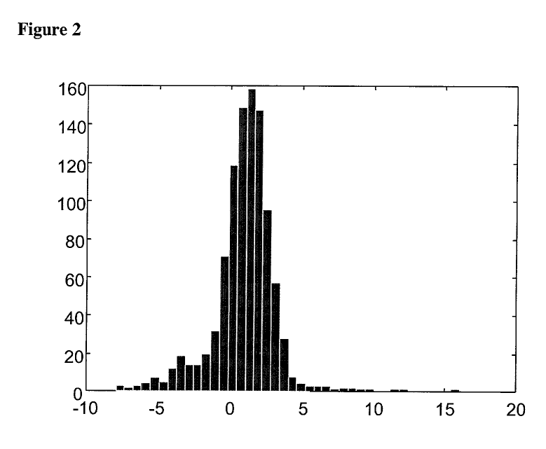

The values of ΔTm were arranged in the form of a histogram presented in FIG. 2. This histogram demonstrates the existence of two classes of complexes formed between HU protein and oligonucleotides. The first, major class of complexes has a positive shift of ΔTm of approximately +3° C. The second class of weak complexes comprising nearly 150 examples has a negative shift of ΔTm of approximately −3° C.

A special analysis to characterize the differences between these two types of complexes was performed. It was found that the A/T content of the duplexes was not the same. The A/T content within the major class has been shown to be 41%, while within the minor class, 62%.

The probability of the presence of one, two, or more A/T pairs in each class of duplexes was calculated, and it was observed that the minor class contains, for the main part, the A/T sequences of four, five, and sometimes six bases pairs, whereas in the major class, the sequences were of two, three, and sometimes four A/T base pairs. These results support at least one simple explanation of the difference between the two classes of complexes. Without limiting the scope of the present invention, it is believed that in the minor class of complexes, HU protein binds to a certain percentage of the single-stranded oligonucleotides, thus, decreasing the melting temperature of the complex. The binding is predominately with long A/T sequences, which are low melting. Again without limiting the scope of the invention, it is believed that in the major class, HU protein binds to double-stranded oligonucleotides and, thereby, increases the Tm.

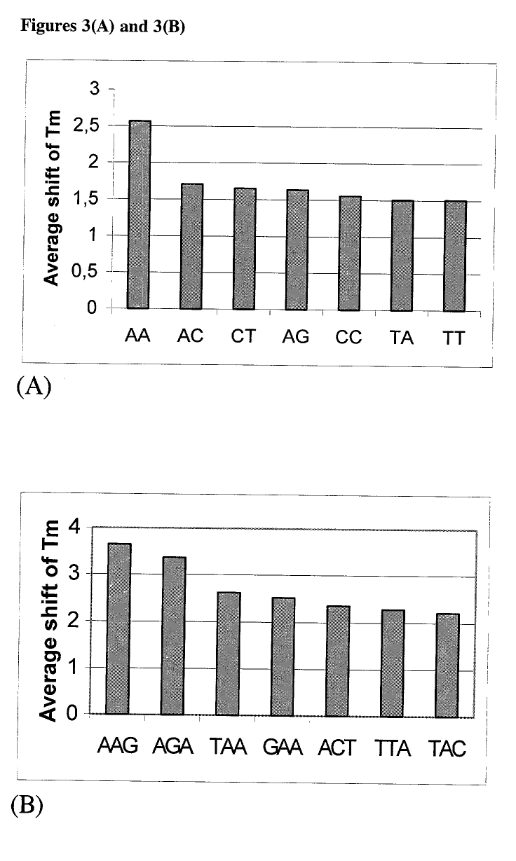

A special study of the specificity of HU protein binding to ds DNA, which complex is known to be non-specific, was carried out. The generic gel-pad microchip provides some additional possibilities for finding motifs in DNA sequences, which may be preferential for protein binding. The total values of ΔTm for the statistical investigation of the specificity of the complexes were used. For all the oligonucleotides of the major class of complexes the average shift in Tm for the sequences containing different motifs was calculated. First, the the average ΔTm for all dinucleotides was calculated. These results are presented in FIG. 3A. The motif AA has the strongest shift of Tm, as compared with the others. The results for three base-pair motifs are presented in FIG. 3B. The motifs AAG, AGA, and, to a lesser extent, TAA are the best. A non-limiting hypothesis that can be derived from these results is that HU protein binding to DNA has a demonstrable preference for some sequence motifs. The specificity of the protein binding to ds DNA is not marked; and only statistical analysis of a large data set could reveal preferential motifs.

Analysis of Fluorescent Signals of HU Protein-DNA Complexes in Comparison with Tm

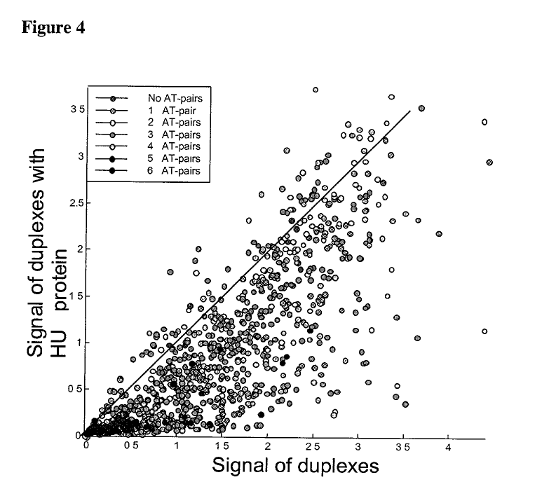

Next the relationship between the melting temperature of the HU protein-oligonucleotide complex and the intensity of fluorescence on the generic microchip was investigated. A correlation between the histogram of Tm values and the pattern of microchip fluorescence in the presence of HU protein was sought. In addition to the data described above it was discovered that the fluorescent signals of some duplexes decreased markedly when HU protein was bound. Thus, the pattern of signals from the microchip was substantially changed when HU protein was applied. The fluorescent signals from the microchip in the presence of HU protein were plotted against the signals obtained when no protein was there. The result obtained is shown in FIG. 4. The G/C-rich duplexes were marked with dark gray, the A/T-rich ones, with black, and the intermediate ones, with light gray.

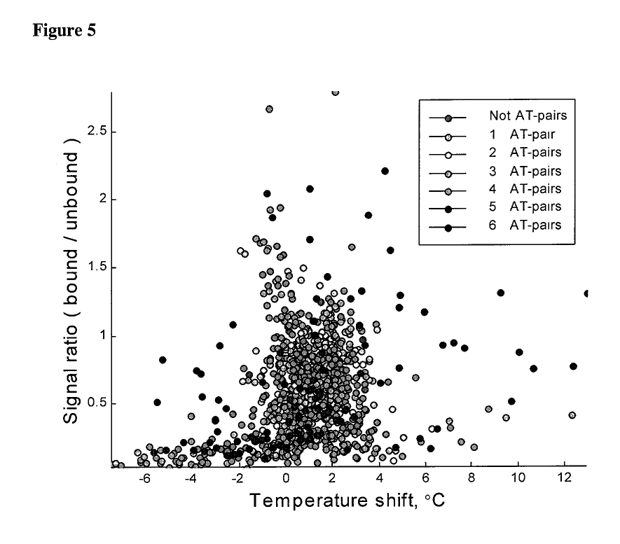

This figure shows that the duplexes where the fluorescent signal is quenched are A/T-rich (black). It was determined that A/T-rich duplexes are presented in the left shoulder of the ΔTm histogram, where the ΔTm is negative, and accordingly proposed that there might be a correlation between ΔTm and the signal quenching dependent on the A/T content of the duplex. This correlation is plotted in FIG. 5. One can see that the pattern created by the A/T-rich duplexes differs from that obtained with the G/C-rich ones. All these G/C-rich duplexes have a positive temperature shift and are not quenched when bound to HU protein. Intermediate duplexes also appear near the center of the graph. However, some A/T-rich duplexes are positioned in the left corner: they have negative temperature shifts and a quenched fluorescent signal.

The main result derived from the data presented in FIGS. 4 and 5 is that the duplexes with different A/T content have different properties both in the Tm shift and for the quenching of fluorescent signal when in complex with HU protein. Without limiting the scope of the present invention, the results obtained support the model that, in the case of the low melting A/T-rich duplexes, HU protein binds DNA via its two single strands and, therefore, decreases the Tm and quenches the fluorescent signal from the gel pad. HU protein is known to bind to ss DNA with a constant of approximately the same order as that for ds DNA.

Binding of HU Protein to Gel-Immobilized Octamers

HU protein is known to bind to ss DNA. In the recent studies ss DNA fragments of 20 to 40 bp, or more, were used to measure the binding constant with HU protein. Oligonucleotides of this length are forced by HU to adopt some secondary structures. In our experiments, gel-immobilized short octamers were used, which, therefore, cannot form any secondary structure, although the present invention is not limited to nucleic acids without secondary structure. Under such conditions, the “basic” constant of HU protein binding to small ss DNA fragments was measured.

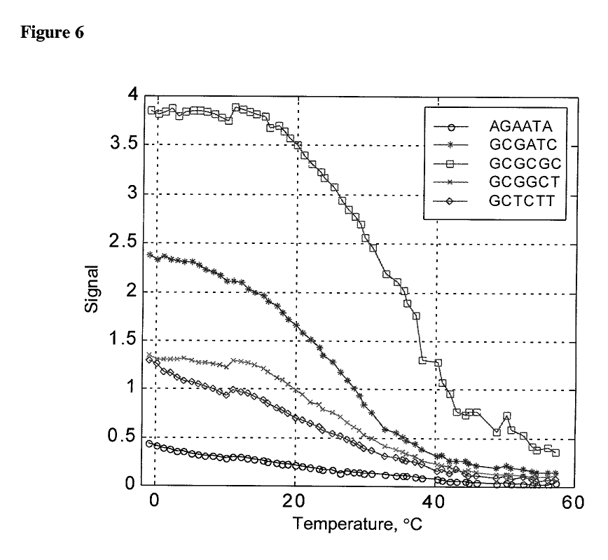

FITC-labeled HU protein was incubated with the microchip containing immobilized octamers as described in Materials and Methods, with the exception that the concentration of NaCl was reduced to 20 μM, since the higher salt concentration was found to weaken the binding of HU proteins to the octamers. The temperature of the microchip was gradually increased, and the process of complex dissociation was monitored by the fluorescence emitted from the FITC-labeled HU protein. Nearly 4,000 melting curves of HU protein-ss DNA complexes were obtained. Some typical dissociation curves are presented in FIG. 6. It can be observed that the dissociation curves of these complexes are not cooperative. This means that one HU protein molecule forms a complex with one immobilized octamer. The dissociation of the complexes was measured, both on the generic microchip containing 4,000 oligonucleotides and on a small “research chip” with only 7 immobilized octamers. All the melting curves obtained were of the same type.

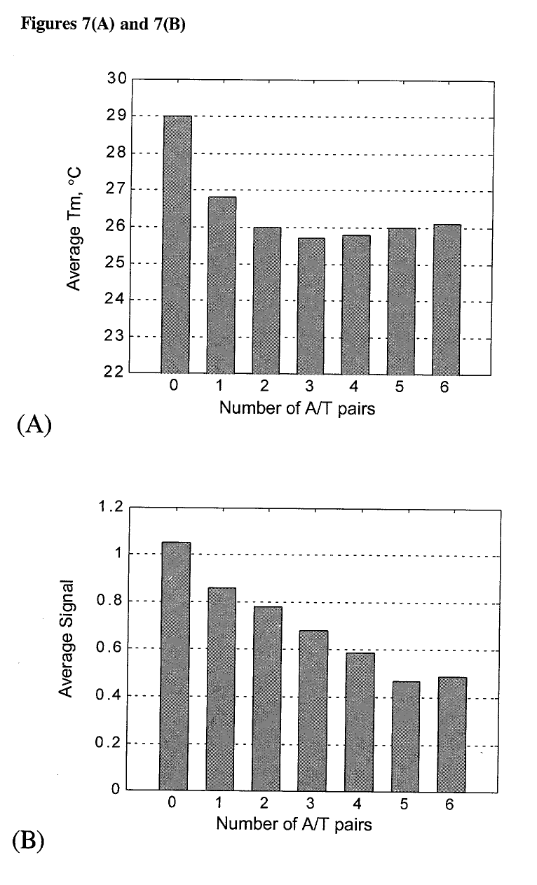

The Tm of HU protein-ss DNA complexes were evaluated, and the values of Tm for 4,000 melting curves were approximated by least squares method using the equation (1) already described. The statistical analysis of the data obtained shows a relatively low specificity of the binding of HU protein to ss DNA. The histogram presented in FIG. 7A shows that the Tm of the complex decreases from 29° C. to 25° C. when the G/C content of the oligonucleotide core decreases from six to four base pairs. All oligonucleotides containing three G/C base pairs, or less, within the hexamer core have the same Tm value. The analysis of the 4-bp motifs demonstrates that GCGC is clearly the strongest sequence for HU binding to ss DNA (data not shown). A similar dependence has been found for the intensity of the fluorescence signal. The histogram shown in FIG. 7B demonstrates that the intensity of the signal gradually lessens with the decrease in number of G/C within the hexamer core of the gel-immobilized oligonucleotides.

Discussion

In the present study, the HU protein-DNA interaction by means of the generic gel-pad microchip was investigated. HU binding to both ds DNA and ss DNA was studied. The large data set obtained enables meaningful statistical analysis of these binding curves; non-limiting conclusions which can be reached are summarized below:

(1) HU protein forms two classes of complexes with DNA, a major one with ds DNA and a minor one with ss DNA. The complexes from the minor class are formed with low melting oligonucleotides and the binding decreases the Tm;

(2) The major class of complexes is formed with ds DNA. In general, it is not specific, but there are some motifs, such as AA, AAG, or GAA, which seem preferred and which, in addition, increase the Tm;

(3) Duplexes with different A/T content have different properties both for shifts of Tm and for quenching of fluorescent signals, when in complexes with HU. The results obtained support the model that in the case of the A/T-rich duplexes, HU protein binds to each single strand of ds DNA, therefore, decreasing the Tm and quenching the fluorescent signal from this gel pad.

(4) HU protein does not have a strong binding specificity for ss DNA fragments, but the binding constant is higher in the case of G/C-rich sequences. GCGC is the best binding motif found among all 4-bp sequences.

It should be recalled that during the first characteristic studies of HU protein, it was observed that this protein associated with the E. coli nucleoid can bind equally well to ds DNA and ss DNA. Rouviere-Yaniv, J. and Gros, F (1975) Proc. Natl. Acad. Sci. USA, 72, 3428-3432. To document the HU-DNA interactions, some studies of the effect of HU protein during the thermal denaturation of λDNA have also been performed Rouviere-Yaniv, J., et al. (1977) In The Organisation and Expression of the Eukariotic Genome, Academic Press, New York, 211-231. These studies showed that the melting of certain AT-rich portions of λDNA happened first. It is very reassuring that the new and much more powerful technology of microchip analysis can confirm, and details, these preliminary data performed a long time ago with more time consuming techniques.

To conclude, the results presented here, demonstrates how the experimental data obtained from generic microchips can be used for statistical computer analysis. This approach offers a way forward for the future studies of the nucleic acid-protein interactions.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” “more than” and the like include the number recited and refer to ranges which can be subsequently broken down into subranges as discussed above. In the same manner, all ratios disclosed herein also include all subratios falling within the broader ratio.

One skilled in the art will also readily recognize that where members are grouped together in a common manner, such as in a Markush group, the present invention encompasses not only the entire group listed as a whole, but each member of the group individually and all possible subgroups of the main group. Accordingly, for all purposes, the present invention encompasses not only the main group, but also the main group absent one or more of the group members. The present invention also envisages the explicit exclusion of one or more of any of the group members in the claimed invention.

All references disclosed herein are specifically incorporated herein by reference thereto.

While preferred embodiments have been illustrated and described, it should be understood that changes and modifications can be made therein in accordance with ordinary skill in the art without departing from the invention in its broader aspects as defined in the following claims.

What is claimed is:

1. A method for characterizing a nucleic acid-protein interaction comprising:
(a) immobilizing a nucleic acid or a protein on a solid support;
(b) contacting the nucleic acid and the protein under conditions which allow the nucleic acid and the protein to interact; and
(c) measuring the strength of the nucleic acid-protein interaction.

2. The method of claim 1 further comprising repeating steps (a) through (c) one or more times.

3. The method of claim 2 wherein the nucleic acid, protein or both used in repeated steps (a) through (c) are different from the respective nucleic acid, protein or both used in the first iteration.

4. The method of claim 1 wherein the nucleic acid is selected from the group consisting of ss RNA, ds RNA, ss DNA, ds DNA and PNA.

5. The method of claim 1 wherein the solid support is a gel pad.

6. The method of claim 1 wherein the strength of the nucleic acid-protein interaction is measured through Tm or a change in Tm.

7. The method of claim 1 wherein the strength of the nucleic acid-protein interaction is measured through fluorescence or a change in fluorescence.

8. The method of claim 1 wherein the nucleic acid sequence is selected from the group consisting of a nucleic acid having a predetermined sequence and nucleic acid not having a predetermined sequence.

9. The method of claim 1 wherein the protein is selected from the group of proteins consisting of a predetermined protein and a protein which is not predetermined.

10. The method of claim 8 wherein the nucleic acid does not have a predetermined sequence further comprising determining the sequence of the nucleic acid.

11. The method of claim 9 wherein the protein is not predetermined further comprising determining the identity of the protein.

12. The method of claim 1 wherein the nucleic acid sequence is a nucleic acid encoding a functional nucleic acid sequence.

13. The method of claim 12 wherein the functional nucleic acid sequence is a promoter or gene.

14. The method of claim 1 wherein the protein modulates the activity or expression of a gene or gene product.

15. A kit for characterizing nucleic acid-protein interactions comprising instructions for carrying out the method of claim 1.

16. The kit of claim 15 further comprising one or more of a solid support, buffer, dyes or disposable lab equipment.

17. A method for characterizing a protein-protein interaction comprising:
(a) immobilizing a protein on a solid support;
(b) contacting the protein with a second protein under conditions which allow the proteins to interact; and
(c) measuring the strength of the protein-protein interaction.

18. The method of claim 17 further comprising repeating steps (a) through (c) one or more times.

19. The method of claim 18 wherein the protein, second protein or both used in repeated steps (a) through (c) are different from the respective protein, second protein or both used in the first iteration.

20. The method of claim 17 wherein the solid support is a gel pad.

21. The method of claim 17 wherein the strength of the protein-protein interaction is measured through fluorescence or a change in fluorescence.

22. A kit for characterizing protein-protein interactions comprising instructions for carrying out the method of claim 17.

23. The kit of claim 22 further comprising one or more of a solid support, buffer, dyes or disposable lab equipment.
